(19) United States
(12) Patent Application Publication
(10) Pub. No.: US 2002/0010510 A1
(43) Pub. Date: Jan. 24, 2002

(54) VARIABLE MODULUS CORNEAL IMPLANT AND FABRICATION METHODS

(76) Inventor: THOMAS A. SILVESTRINI, ALAMO, CA (US)

Correspondence Address: Antoinette F Konski, McCutchen Doyle Brown & Enersen LLP, Three Embarcadero Center, Suite 1800, San Francisco, CA, 94111-4067, US

(21) Appl. No.: 09/185,913

(22) Filed: Nov. 4, 1998

(51) Int. Cl. A61F 2/14

(52) U.S. Cl. 623/5.12; 623/5.16

(57) ABSTRACT

An intracorneal implant has a geometry bounded by a ring-shaped section of a cone, approximating a section of a sphere. The intracomeal implant has at least one region of modified elasticity. Typically, the intracorneal implant will have two or more regions of modified elasticity circumferentially spaced around the implant. The regions of modified elasticity affect the cone angle of the implant within each region and hence the corrective power of each region on the cornea. The intracorneal ring implant can be used for correction of various refractive defects of the vision, in particular, astigmatism or astigmatism combined with myopia or hyperopia. The implant may be formed as a ring, split ring, gapped ring or one or more segments of a ring.

### BACKGROUND OF THE INVENTION

The present invention relates generally to an improved device and surgical method for correcting defects in vision. More particularly, it relates to an intracorneal implant for correcting various refractive defects of the vision, in particular, astigmatism or astigmatism combined with myopia or hyperopia.

In order to fully understand the present invention it is important to have an understanding of the function of the eye and the various defects which can effect the vision. Ametropia, which is responsible for various refractive defects of the vision, is caused by a discrepancy between the refractive power of the eye and the dimensions of the eye, such that images are not brought into proper focus on the retina. Forms of ametropia include myopia, hyperopia and astigmatism. In the normal or emmetropic eye, light rays from a distant object which enter the eye parallel to the optical axis are focused directly on the retina resulting in a clear image of distant objects. The light rays are focused by the combined refractive power of the cornea and the crystalline lens of the eye. The light rays are first refracted at the anterior surface of the cornea, then refracted again at each interface between the cornea, the aqueous humor, the crystalline lens and the vitreous humor. Since the greatest difference in refractive index is at the interface between the cornea and the air, most of the refraction occurs at the anterior surface of the cornea. Light rays from near objects reach the eye at a divergent angle. The diverging light rays would normally be focused at a point behind the retina, resulting in an unfocused image of near objects. However, the eye brings the image into clear focus by accommodation of the crystalline lens through the action of the ciliary muscles which surround the crystalline lens. Accommodation results in a thickening of the crystalline lens which increases its degree of curvature and therefore its refractive power so that the image is brought to a sharp focus on the retina. The amplitude of accommodation of the crystalline lens determines how close objects can be and still be focused sharply on the retina. The closest distance at which the eye can still bring an object into focus is, called the near point of distinct vision.

Myopia or nearsightedness is a form of ametropia caused by a mismatch between the refractive power of the eye and the dimensions of the eye that results in light rays entering the eye parallel to the optical axis being focused in front of the retina. Axial myopia is caused by the anteroposterior axis of the eye being too short, while curvature myopia is caused by excessive convexity of the refractive surfaces of the cornea and/or the lens. In the myopic eye, light rays from a distant object which enter the eye parallel to the optical axis are focused at a point in front of the retina. By the time the light rays have reached the retina, they have already diverged somewhat, resulting in an unfocused image of distant objects. On the other hand, the diverging light rays from near objects can be brought into sharp focus on the retina, with little or no accommodation of the crystalline lens, depending on the degree of myopia. With full accommodation of the crystalline lens, the myopic eye can focus light rays from objects that are very close to the eye. The near point of distinct vision is very close to the eye, hence the term nearsightedness.

Nearsightedness has traditionally been treated with negative power corrective lenses, either with spectacles or contact lenses, which diverge the light rays somewhat before they reach the eye, resulting in normal, clear vision at all distances.

Hyperopia or farsightedness is a form of ametropia caused by a mismatch between the refractive power of the eye and the dimensions of the eye that results in light rays entering the eye parallel to the optical axis being focused behind the retina. Axial hyperopia is caused by shortness of the anteroposterior axis of the eye, while curvature hyperopia is caused by insufficient convexity of the refractive surfaces of the cornea and/or the lens. In the hyperopic eye, light rays from a distant object which enter the eye parallel to the optical axis are focused at a point behind the retina, which would normally result in an unfocused image of distant objects. However, with accommodation of the crystalline lens, the eye can bring the image into sharp focus on the retina for clear vision of distant objects. For near objects, the hyperopic eye focuses the diverging light rays which enter the eye at a point very far behind the retina. The hyperopic eye attempts to bring the image into focus through accommodation of the crystalline lens. However, because there is a limit to the amplitude of accommodation possible for the crystalline lens, the point of focus for near objects still falls behind the retina, resulting in an unfocused image. The near point of distinct vision that can be accomplished through full accommodation of the crystalline lens is farther removed from the eye, hence the term farsightedness. Farsightedness has traditionally been treated with positive power corrective lenses, either with spectacles or contact lenses, which converge the light rays somewhat before they reach the eye, resulting in normal, clear vision at all distances.

Alternatively, because the eye can accommodate sufficiently for distant vision without corrective lenses, moderate amounts of hyperopia are sometimes treated with “reading glasses” which are only needed for viewing objects closer than the near point of distinct vision.

Astigmatism is a form of ametropia caused by the radius of curvature of the refractive surfaces of the cornea and/or the lens of the eye in one plane being longer or shorter than the radius of curvature in a plane at right angles to it. As a result, rays of light entering the eye are not focused at a sharp point on the retina, but are spread over a diffuse area. Astigmatism can occur in combination with myopia, hyperopia or presbyopia. Astigmatism is traditionally treated with toric corrective lenses that have greater or lesser refractive power in one plane than in the plane at right angles to it. A negative power correction for myopia or a positive power correction for hyperopia can be superimposed on the toric correction for astigmatism. Astigmatism is usually corrected with spectacles, however some degree of success has been achieved for correcting modest amounts of astigmatism with toric contact lenses.

In addition to the traditional approach of correcting ametropia with corrective lenses, various surgical methods for vision correction are also known. Recognized surgical methods include radial keratotomy, exemplified by U.S. Pat. No. 4,688,570 granted to Kramer et al., entitled Ophthalmologic Surgical Instrument, and U.S. Pat. No. 4,815,463 granted to Hana, entitled Surgical Device for Radial Keratotomy, and photorefractive keratectomy, exemplified by U.S. Pat. No. 4,941,093 granted to Marshall et al., entitled Surface Erosion Using Lasers, and U.S. Pat. No. 5,163,934 granted to Munnerlyn, entitled Photorefractive Keratectomy. In radial keratotomy and photorefractive keratectomy, the cornea of the eye is reshaped by cutting or by laser ablation to correct vision defects. These surgical approaches have significant drawbacks in that both methods involve substantial trauma to the cornea of the eye from multiple incisions or laser ablations in or near the optical zone of the cornea. Such trauma can result in the formation of scar tissue, which, if it extends into the optical zone of the cornea, can interfere with the patient's vision. Also, in a small percentage of cases, the results of the surgery are unsatisfactory and can even cause a deterioration of the patient's vision instead of an improvement. Unfortunately, the effects of radial keratotomy and photorefractive keratectomy are irreversible so the patient must accept the outcome of the surgery if it is unsuccessful.

Another surgical approach for treating refractive defects of the vision involves the use of corrective implants surgically implanted into the cornea of the eye. One variant of this surgical approach is to implant a corrective lens directly into the optical zone of the cornea to correct the patient's vision. A second variant of this surgical approach involves the use of intracorneal implants implanted for affecting the actual curvature of the corneal surface.

For example, U.S. Pat. No. 4,655,774 granted to Choyce for an IntraCorneal Implant for Correction of Aniridia describes a method for implanting an artificial iris with an optional corrective lens within the cornea of the eye for correcting vision defects. The surgical method described involves making an incision into the cornea, creating a pocket within the cornea using a curved dissecting instrument, inserting the implant into the pocket and closing the incision. The method requires an incision at least as large as the diameter of the rigid implant (estimated to be about 6-8 mm) and the pocket forming step does not provide positive control of the margins of the pocket formed. These aspects of the surgical procedure may inhibit healing of the cornea after implantation of the device.

U.S. Pat. No. 5,196,026 granted to Barret et al. for a Method of Implanting Corneal Inlay Lenses Smaller Than the Optic Zone describes a surgical method that involves making an incision near the edge of the cornea the size of the lens to be inserted and creating a pocket to the center of the cornea using a blunt spatula. A circular or ring-shaped lens 2-4 mm in diameter is inserted into the pocket. This method allows a smaller incision than Choyce, but only because the actual implant is smaller. Using this method for larger implants to affect the entire optical zone would naturally require a larger, more traumatic incision. This method also does not provide positive control of the margins of the pocket formed.

U.K. patent GB 2,095,119 granted to Tennant et al. for a Circular Keratotomy with Insert for Myopia Correction describes a surgical method wherein the epithelial layer of the cornea is removed and the optical zone is circumscribed with a circular groove which causes the cornea to flatten. A circular insert is implanted within the groove to maintain the space while scar tissue grows to cover the insert and the epithelium regrows over the corneal surface. This method involves considerable trauma to the eye in that it requires removal of the epithelial layer and a large circular incision around the optical zone. Because of the amount of scar tissue produced, this procedure would not be reversible without substantial trauma to the corneal tissues.

U.S. Pat. No. 4,976,719 granted to Siepser for a Device used to Change Corneal Curvature describes a similar ring-shaped corneal implant that includes the improvement of a turnbuckle connector which allows the size of the implant to be adjusted in order to correct for myopia or hyperopia. The surgical procedure for implanting the ring-shaped implant avoids the need for a circular incision by inserting one end of the ring through a puncture in the cornea and advancing it in a circular path between the corneal layers. However, a 4 to 5 mm incision is still required for manipulation of the turnbuckle. In addition, there is no positive control of the path of the ring wire as it is advanced through the corneal tissue.

U.S. Pat. No. 5,391,201 granted to Barret et al. for a Method of Using a Corneal Ring Inlay describes a surgical method of implanting a continuous ring into the cornea that involves either a peripheral incision in the cornea followed by undermining the cornea in a circular fashion or slicing the top of the cornea off completely. Either of these surgical approaches is highly traumatic to the corneal tissue and would inhibit healing of the cornea. The consequent scarring would likely make this procedure irreversible.

U.S. Pat. No. 4,452,235 granted to Reynolds for a Method for Corneal Curvature Adjustment describes a surgical method for implanting a split ring within the cornea for correcting vision defects. A 1 mm incision is made in the cornea and a circular dissecting tool is used to create a circular path within the cornea back to the incision point. One end of the split ring is connected to the dissecting tool and the tool is backed out, pulling the split ring in behind it. Once the split ring is inside the cornea, it is detached from the tool and the diameter of the ring is adjusted to correct the patient's vision, then the ends of the ring are fixed together. This method creates a very small incision in the cornea which promotes healing. However, the incision is directly over the path of the split ring implant, particularly the ends of the ring, so that there may be stress on the incision that could interfere with healing and may increase scar tissue formation.

U.S. Pat. No. 5,405,384 granted to Silvestrini describes an Astigmatic Correcting Intrastromal Corneal Ring having two or more regions of increased bulk spaced around the ring for correction of astigmatism.

International patent application WO 95/03747 by Silvestrini describes a Segmented Pliable Intrastromal Corneal Insert for correction of astigmatism.

U.S. Pat. No. 4,799,931 granted to Lindstrom discloses an intracorneal lens having through holes which are filled with a gas permeable polymer or metabolite permeable material, a biocompatible polymer or biocompatible material, or a neutral or negatively charged material. The filled holes are provided to allow the passage of gases, fluids and nutrients therethrough.

U.S. Pat. No. 4,624,669 granted to Grendahl discloses a corneal inlay made of polysulfone or PMMA and having a plurality of small holes to pass nutrients and fluids from the bottom surface of the cornea to the top surface of the cornea.

U.S. Pat. No. 5,628,794 granted to Lindstrom describes a multifocal corneal implant lens having a hydrogel coating and nutrient transmission holes which are filled with permeable hydrogel material, purportedly to prevent the growth of scar tissue within the holes.

U.S. Pat. No. 4,851,003 granted to Lindstrom describes an intracorneal or epicorneal lens having a plurality of fixation holes placed about the outer circumference of the lens hydrogel coating and nutrient transmission holes which are filled with permeable hydrogel material, purportedly to prevent the growth of scar tissue within the holes.

International patent application WO 97/28759 by Silvestrini describes an intracorneal insert for effecting a change in the corneal curvature to treat conditions such as astigmatism, myopia and hyperopia. The insert includes one or more arcuate or arc-shaped segments. The extent of corneal correction is a function of one or more of arc angle, radius of curvature, cone angle, modulus of elasticity and thickness of the segment. Astigmatism is treated by use of segments having varying thicknesses.

International patent application WO 97/49354 by Hennig describes a corneal ring, for correction of the refractive power of the eye, which consists of a ring or ring segment of metal or plastic which is adjustable after insertion by the action of electromagnetic radiation or magnetic treatment.

In view of the above approaches to vision correction, there is a continuing need to provide an improved device and associated surgical method for correcting astigmatism and other refractive defects of the eye using an intracomeal implant. It is desirable that such a method provide a permanent, but reversible, correction of vision defects without substantial trauma to the corneal tissue.

### SUMMARY OF THE INVENTION

“Bulk modulus” of elasticity, as used herein, refers to an inherent property of a homogeneous material, i.e., the modulus of elasticity of the material independent of its cross sectional geometry.

“Apparent modulus” of elasticity, as used herein, refers to an inherent property of a composite material. The apparent modulus of elasticity of a composite material refers to the modulus of elasticity from a composite macromechanical point of view, i.e., the material is presumed homogeneous and the effects of the constituent materials are detected only as averaged apparent properties of the composite. That is, the bulk modulus of elasticity of each constituent material making up the composite is combined to form an apparent modulus of the material.

“Effective modulus” of elasticity, as used herein, refers to a stiffness or elasticity of an object taking into consideration the cross sectional geometry, to include thickness variations, etc. Thus, shape and moment of inertia effect the effective modulus of elasticity in addition to the factors that effect bulk and apparent moduli, as discussed above.

The device of the present invention provides an intracorneal implant that has a geometry which can be at least partially bounded by a ring-shaped section of a cone approximating a section of a sphere. The cross section of the implant may be circular, elliptical, hexagonal or a number of other polygonal shapes. Additionally other cross-sectional shapes which approximate a polygonal shape but are asymmetric, may be used. The intracorneal implant according to the present invention has at least one region of modified elasticity. Typically, the intracorneal implant will have one or more regions of modified bulk or apparent modulus of elasticity spaced around the implant. The regions of modified bulk or apparent modulus of elasticity affects the ability of the implant to maintain a predetermined cone angle of the intracorneal implant within each region and hence the corrective power of each region on the cornea. That is, by modifying regions, the ability of the implant to maintain a given angle, or to push back against a load applied thereto by the corneal tissue, once implanted, may be accurately controlled. In the method of the present invention the intracorneal implant is implanted into the cornea of the eye for correction of various refractive defects of the vision, in particular, astigmatism or astigmatism combined with myopia or hyperopia.

In a first aspect of the invention, the intracorneal implant is made in the shape of a ring, with four sector-shaped regions, including first and third regions made of a relatively stiff, high elastic modulus material and second and fourth regions made of a flexible, low elastic modulus material. The regions may be joined together by radially oriented butt joints, tapered joints or lap joints, or by butt joints or lap joints arranged along two parallel lines. The intracorneal ring implant may be slit to form a split ring or it may be formed as a continuous ring, or it may be formed with a gap, depending on the chosen surgical technique for implantation. If the intracorneal ring implant is made as a continuous ring, the second and fourth regions of the implant are preferably made flexible enough that the implant can advantageously be folded for insertion through a small incision into the cornea.

As an additional alternative, the implant may be formed as two or more segments which, when properly oriented, approximate the shape of a ring. Each segment may have one or more regions of modified bulk and/or apparent modulus of elasticity. On the other hand one or more segments may have consistent elasticity throughout, but have a different bulk and/or apparent modulus of elasticity than at least a portion of another segment to be implanted with it.

In a second aspect of the invention, the first and third regions of the intracorneal ring implant (or non-adjacent regions of segments) are made of a relatively stiff composite material with a high apparent modulus of elasticity and the second and fourth regions (or other non-adjacent regions of segments) are made of a flexible, homogenous material with a relatively low bulk modulus of elasticity. In one exemplary embodiment, this is accomplished by reinforcing the first and third regions of a flexible ring-shaped implant body with rigid sectorshaped inserts.

In a third aspect of the invention, the varied stiffness of the different regions is created by changing the internal geometry of the intracorneal implant without modifying the thickness or the cross sectional geometry of the implant. In one exemplary embodiment, this is accomplished by piercing the second and fourth regions of a ring-shaped implant body (or non-adjacent regions of segments) with an array of holes, torturous cavities, filled holes, bubbles, or the like, to render them more flexible than the first and third (or other non-adjacent) regions, i.e., by changing the apparent modulus of elasticity. In another exemplary embodiment, this principle is used to create an intracorneal ring implant where the entire ring-shaped implant body or segments is/are pierced with an array of holes, torturous cavities, partial cuts, filled holes, bubbles, or the like, to modify the apparent modulus of elasticity of the entire implant, albeit, not necessarily equally. That is, a first segment might have, for example, a thirty percent hole volume fraction or filled hole volume fraction (i.e., hole or filled hole volume compared to the total volume of the first segment), while an adjacent second segment might have a fifteen percent hole volume fraction or filled hole volume fraction. This of course would result in the two segments having different characteristics of flexibility, due to their different apparent moduli of elasticity.

### BRIEF DESCRIPTION OF THE DRAWINGS

FIG. 1 shows an eye of a patient with an intracomeal ring implant within the cornea of the eye.

FIG. 2 is a cross section of the cornea and the intracorneal ring implant of FIG. 1 taken along section line 2-2.

FIG. 3 is a cross section of the cornea and the intracorneal ring implant of FIG. 1, taken along section line 3-3.

FIG. 4 shows a plan view of an embodiment of the intracorneal ring implant having four regions of varying elasticity joined by butt joints or casting.

FIG. 5 shows a perspective view of the intracorneal ring implant of FIG. 4.

FIG. 6 shows a plan view of an embodiment of the intracorneal ring implant having four regions of varying elasticity joined by lap joints.

FIG. 7 shows a perspective view of the intracorneal ring implant of FIG. 6.

FIG. 8 shows a plan view of a continuous ring embodiment of the intracorneal ring implant.

FIG. 9 shows a perspective view of the continuous ring intracorneal ring implant of FIG. 8.

FIG. 10 shows the continuous ring intracorneal ring implant of FIGS. 8 and 9 folded for implantation into a pocket formed within the cornea.

FIG. 11 shows a plan view of an embodiment of the intracorneal ring implant having four regions of varying elasticity joined together along two sets of parallel lines.

FIG. 12 shows a perspective view of the intracorneal ring implant of FIG. 11.

FIG. 13 illustrates a manufacturing process for the intracorneal ring implant of FIGS. 11 and 12.

FIG. 14 shows a plan view of an embodiment of the intracorneal ring implant with two regions of modified elasticity made of a layered composite.

FIG. 15 shows a perspective view of the intracorneal ring implant of FIG. 14.

FIG. 16A shows a plan view of an embodiment of the intracorneal ring implant with two regions of modified elasticity created by altering the internal geometry of the regions.

FIG. 16B shows a plan view of one of many possible alternative arrangements to the embodiment shown in FIG. 16A.

FIG. 17 shows a perspective view of the intracomeal ring implant of FIG. 16.

FIG. 18 shows a plan view of an embodiment of the intracorneal ring implant where the entire ring-shaped body has modified elasticity created by altering the internal geometry of the ring.

FIG. 19 shows a perspective view of the intracorneal ring implant of FIG. 18.

FIG. 20 shows an eye of a patient with intracorneal ring segments implanted within the cornea of the eye.

FIG. 21 shows a partial cross sectional view of a tapered joint between sections having different moduli of elasticity.

FIG. 22 shows irregularly, but symmetrically formed joints at the intersections of portions of an implant.

FIG. 23 shows an example of an implant in which portions have been cocasted.

FIG. 24 illustrates an implant made according to a photomask technique, as described herein.

FIG. 25 illustrates an implant made according to a multiple masking technique.

FIG. 26 is a cross-sectional view of a cornea showing portions of an implant having matching cone angles.

FIG. 27 is a cross-sectional view of a cornea showing portions of an implant having mismatching cone angles greater than the matching cone angles.

FIG. 28 is a cross-sectional view of a cornea showing portions of an implant having mismatching cone angles less than the matching cone angles.

### DETAILED DESCRIPTION OF THE INVENTION

An intracorneal implant according to the present invention may be in the form of a ring, a split ring, a gapped ring, segments of a ring or radial implants, including composite radial implants having a segment of a ring. The following are representative examples of preferred procedures for implanting various forms of the implant according to the present invention.

Continuous Rings

Initially, the geometric center of the cornea is marked with a blunt instrument (e.g., a Sinskey hook) using an operating microscope for fixation. A zone marker (e.g., an 11 mm zone marker) can be used to aid in locating the center point. A sterile marking pen may be used to enhance the mark. This center mark is used as the reference point throughout the surgical procedure.

Next, the contact surface of an incision and placement marker is marked, using a sterile marking pen, for example. The incision and placement marker is then centered on the center mark created at the geometric center described above, by lining up the reticle of the incision and placement marker with the center mark. The contact surface of the marker is contacted lightly against the cornea, making an inked marking of where the radial (or, alternatively, circumferential) incision will be made. Also, an indication of where the outer edge of the ring will be placed is made. A visual verification is made that the mark is at least 1 mm from the limbus. If the mark is too close to the limbus, re-marking of the geometric center of the cornea is required, to get closer to the actual geometric center.

A pachymetry measurement is made to determine the thickness of the corneal tissue at the incision site. Next, a calibrated, diamond knife is set to 0.430 mm (430μ) or 68% of the intraoperative pachymetry reading taken at the incision site. The diamond should either have an angled cutting edge of 15° or less, or have a rectangular blade of 1 mm width or less.

A radial incision is made by tracing to the outside edge of the incision mark. Alternatively, a circumferential incision may be made. The incision may also be made at an oblique angle to the surface of the cornea. The incision length may range from about 0.7 to 1.8 mm, and is preferably about 1.2 mm. Special care should be taken to ensure that the incision is kept approximately 1 mm away from the limbus. The incision area is then thoroughly irrigated with balanced salt solution after completing the incision. A Merocel® spear or equivalent is used to remove any loose epithelial cells and excess balanced salt solution from the edges of the incision. The epithelium may be rolled away from the incision edges. The incision is again thoroughly irrigated with balanced salt solution prior to any instrument insertion.

A specialized pocketing tool, such as those described in co-pending U.S. application titled “CORNEAL POCKETING TOOL”, filed on Dec. 18, 1997 (attorney docket no. 25169-20053.00), is next used to separate the stromal layers at the appropriate depth at the base of the incision. Co-pending U.S. application titled “CORNEAL POCKETING TOOL”, filed on Dec. 18, 1997 (attorney docket no. 25169-20053.00) is hereby incorporated by reference thereto in its entirety. After placing the pocketing tool instrument in the incision, the pocketing tool is rotated to create an intrastromal separation or pocket.

The pocket may be enlarged as desired using a stromal spreader such as is described in co-pending U.S. application Ser. No. 08/896,792 filed on Jul. 18, 1997 titled “OPTHALMOSURGICAL INSTRUMENTS AND METHODS OF USE”. U.S. application Ser. No. 08/896,792 is hereby incorporated by reference thereto in its entirety. The tip of the spreader is inserted vertically down into the incision until it contacts the bottom of the incision. A blunt dissection or enlarged pocket is then created on one side of the base of the incision by carefully rotating the blade of the spreader instrument within a single stromal plane. The procedure is then repeated on the other side of the incision base. The resultant pockets should be at the same depth as the incision base, as wide as the full incision length, and extend to the full length of the spreader tip.

Corneal thickness gauges, such as those described in co-pending U.S. patent application Ser. No. 08/676,377, for example, may be used to estimate the depth of both pockets. Co-pending U.S. patent application Ser. No. 08/676,377 is hereby incorporated by reference thereto in its entirety. If the pockets are not deep enough in the corneal stroma, the incision is made slightly deeper with the diamond knife and a second pocket or set of pockets are then created at a deeper level with the pocketing tool and spreader in the manner described above.

The incision and placement marker is next indexed into a vacuum centering guide (VCG) such as those described in Loomas, U.S. Pat. No. 5,403,335 or pending U.S. patent application Ser. No. 08/796,595 for example. Both U.S. Pat. No. 5,403,335 and pending U.S. patent application Ser. No. 08/796,595 are hereby incorporated by reference thereto in their entireties. The reticle is aligned with the center mark to center the VCG on the center mark. The VCG is then lowered to contact the sclera of the eye while maintaining centration, and then vacuum is slowly applied. Placement of the VCG over the incision and placement marker, together with proper alignment of the marker on both the center mark and the actual incision, ensure that a window in the VCG is centered about the incision site. The vacuum should start in the range of 12-15 inches of Hg. Once a vacuum seal has been established, a confirmation that the VCG is properly placed is made, by checking centration. If the VCG is not properly positioned, the vacuum must be released, and the above procedure regarding the VCG must be repeated. If the VCG is determined to be properly positioned, the vacuum is then slowly increased to 18-20 inches of Hg. It is recommended that the vacuum not exceed 22 inches of Hg. Next, the incision and placement marker is removed from the VCG.

While maintaining the position of the VCG, a counterclockwise (CCW) dissector, such as that described in copending U.S. application Ser. No. 08/676,377, for example, is inserted into the VCG. The dissector body should be rotated until the tip of the dissector blade is adjacent to the incision site. A counterclockwise glide, such as described in U.S. application Ser. No. 08/896,792, for example, is inserted in the incision, at least 1 mm into the pocket and the dissector tip is rotated under the foot of the glide. Counterclockwise rotation of the dissector body allows the dissector tip to enter the pocket underneath the glide. The dissector blade is then advanced approximately 1 mm to 2 mm, then stopped. The glide is removed while leaving the dissector tip in position in the pocket.

While holding the VCG vertically with one hand, the dissector is rotated counterclockwise from the incision to create a stromal channel. Rotation of the dissector in a counterclockwise direction is continued until the support spoke of the dissector blade contacts the incision edge. Then the dissector blade is removed from the channel by rotating the dissector body clockwise until the dissector tip exits the channel. The dissector is then removed from the VCG.

While maintaining the position of the VCG, a clockwise (CW) dissector, such as that described in copending U.S. application Ser. No. 08/676,377, for example, is inserted into the VCG. The dissector body is rotated until the tip of the dissector is adjacent to the incision site. Next, a clockwise (CW) glide, such as described in U.S. application Ser. No. 08/896,792, for example, is inserted at least 1 mm into the opposite pocket and the dissector tip is rotated under the foot of the glide. Clockwise rotation of the dissector body drives the dissector tip into the pocket. The dissector tip should be inserted underneath the glide foot to enter the pocket. The glide blade is next advanced approximately 1 mm to 2 mm, then stopped in its position. The glide is removed while leaving the dissector tip in position in the pocket.

While holding the VCG vertically with one hand, the dissector is rotated clockwise from the incision to create a second stromal channel. The clockwise rotation of the dissector is continued until the support spoke of the dissector blade contacts the incision edge or until an abrupt decrease in resistance to continued dissection is felt. If the two stromal channels meet upon formation of the second stromal channel, there will be an abrupt decrease in resistance to continued dissection. Then the dissector blade is removed from the channel by rotating the dissector body counterclockwise until the dissector tip exits the channel. The dissector is then removed from the VCG.

If, upon contact of the support spoke of the dissector blade with the incision edge, an abrupt decrease in the resistance has not yet been felt, this generally means that the stromal channels have been formed at different depths and have not met, joined or connected. In this case, a clockwise probe, such as that described in copending U.S. application Ser. No. 08/676,377, for example, is inserted into the clockwise channel. If breakthrough occurred, the probe tip will easily rotate past 200° clockwise rotation. Optimally, the probe will rotate past 200° and clearly into the channel created by the counter-clockwise dissector. If, however, the probe does not rotate past 200°, it will be possible to determine which channel, the clockwise or the counter-clockwise channel is the lower channel. Clockwise and counter-clockwise probes, such as those described in copending U.S. application Ser. No. 08/676,377, for example, are inserted into the clockwise and counter-clockwise channels, respectively. The lower channel is determined by observing the position of the probes at the point where their tips overlap. Once this has been determined, the probes are removed from the channels by rotating them clockwise for the counter-clockwise probe and counterclockwise for the clockwise probe.

If it has been determined that the clockwise channel is the lower one, a clockwise channel connector,, such as that described in copending U.S. application Ser. No. 08/676,377, for example, is inserted clockwise into the clockwise channel and rotated clockwise until it is observed to break through into the counter-clockwise channel or until it has rotated about 330°. If the channels are connected, there will have been an abrupt decrease in resistance to continued dissection. If this is the case, the clockwise channel connector is then rotated counter-clockwise and removed from the clockwise channel. If the channels do not meet, a clockwise finish channel connector, such as that described in copending U.S. application Ser. No. 08/676,377, for example, is inserted into the lower, clockwise channel and rotated clockwise until the lower channel connects with the upper channel, or until the finish channel connector tip rotates through to the entry incision. Channel connecting may be determined when an abrupt decrease in resistance to continued dissection is observed. Once the clockwise and counter-clockwise channels have been connected, the finish channel connector is removed.

If it has been determined that the counter-clockwise channel is the lower one, a counter-clockwise channel connector, such as that described in copending U.S. application Ser. No. 08/676,377, for example, is inserted counter-clockwise into the counter-clockwise channel and rotated counter-clockwise until it is observed to breakthrough into the clockwise channel or until it has rotated about 330°. If the channels are connected, there will have been an abrupt decrease in resistance to continued dissection. The counter-clockwise channel connector is then rotated clockwise and removed from the counter-clockwise channel.

If the channels do not meet, a counter-clockwise finish channel connector, such as that described in copending U.S. application Ser. No. 08/676,377, for example, is inserted into the lower, counter-clockwise channel and rotated counter-clockwise until the lower channel connects with the upper channel, such that is there is an abrupt decrease in resistance to continued dissection or until the finish channel connector tip rotates through to the entry incision. Once the clockwise and counter-clockwise channels have been connected, the finish channel connector is removed.

Alternatively, a complete circular interlamellar pathway can be created using a single circular dissector tool, such as described in copending U.S. application Ser. No. 08/993,946, for example. Copending U.S. application Ser. No. 08/993,946, entitled “Corneal Implant Methods and Pliable Implant Therefor”, filed on Dec. 18, 1997, is hereby incorporated by reference thereto in its entirety. The dissector is inserted and rotated in much the same manner described above with respect to the clockwise and counterclockwise dissectors.

The circular interlamellar pathway next may be optionally expanded radially inward in a controlled stepwise fashion to create a wider intracorneal channel. Preferably, this is done by introducing a channel-widening dissector tool with a side leg ending in a blunt dissecting tip, such as described in copending U.S. application Ser. No. 08/993,946, for example, through the incision and moving the channel-widening dissector tool in an arc-shaped path around the circular interlamellar pathway to widen the intracorneal channel. As with the initial circular interlamellar pathway, the widened intracorneal channel can be created with a single 350 degree circular channel-widening dissector tool, or using a pair of clockwise and counterclockwise 180-200 degree semicircular channel-widening dissector tools. In either case, the tools are preferably designed to interfit with and be guided by the VCG.

Channel-widening dissector tools with progressively longer side legs, such as described in copending U.S. application Ser. No. 08/993,946, for example, are used to expand the channel until the channels merge to form a substantially circular intracorneal pocket. Preferably, a pocket-forming dissector tool, such as that disclosed in co-pending U.S. application Ser. No. 08/993,946, for example, with a side leg that is slightly longer than the radius of the initial circular interlamellar pathway is inserted through the incision into the widened intracorneal channel and the pocket-forming dissector tool is rotated about a central axis to create the intracorneal pocket. The intracorneal pocket can be created with a single 350 degree dissector tool , or using a pair of clockwise and counterclockwise 180-200 degree semicircular dissector tools.

The vacuum is next released and the VCG is removed from the eye. Any stromal debris from the incision site is removed and the incision area is again thoroughly irrigated, using balanced salt solution, prior to insertion of the implant. Optionally, a small amount of Celluvis® or an equivalent lubricating agent may be applied to the surface of the cornea, to avoid direct contact of the implant with the epithelium, although this is not preferred.

Alternatively, the intracorneal pocket can be completed by inserting a curved, blunt dissecting spatula, such as that disclosed in co-pending U.S. application Ser. No. 08/993,946, for example, or similar probe through the incision and dissecting the lamellae across the optical zone of the cornea. Because the outer boundary of the intracorneal pocket has already been carefully and precisely defined by the circular interlamellar pathway, there is less concern about creating irregular edges at the margin of the pocket with the dissecting spatula or of overshooting and dissecting the cornea into the limbus which could cause healing problems for the cornea. Although not preferred, the intracorneal pocket can be formed without the widening steps, using only the initial dissectors and the spatula.

The ring is folded in half and inserted through the incision and into the intracorneal pocket. Once fully inserted, the ring is unfolded and positioned around the optical zone of the cornea.

The folded continuous ring intracorneal implant may be inserted in a number of ways. A standard pair of forceps may be used to grip the implant a small distance away from the incision and advance the implant towards the incision and into the pocket in a series of small increments. Another technique of introducing the implant is to insert the ring into the intracorneal pocket by assembling the implant into a tube element, such as that disclosed in co-pending U.S. application Ser. No. 08/993,946, for example, leaving an end portion of the implant extending from the tube, inserting at least a portion of the tube containing the implant into the pocket, and then using a hook instrument, such as that disclosed in co-pending U.S. application Ser. No. 08/993,946, for example, to pull the ring from the tube. The tube is inserted a distance into the pocket through the incision. The hook instrument is then inserted through the incision and manipulated to engage the portion of the implant extending from the tube. The hook instrument is then advanced towards the incision to pull the implant from the tube. The tube is then withdrawn from the cornea. Alternatively, the ring implant may be pulled from the tube by way of an additional incision on the opposite side of the cornea.

Another tube which may be used for implantation of the ring includes a split to allow for assembly of the implant into the tube. Also, a pushing device, such as plunger, such as that disclosed in co-pending U.S. application Ser. No. 08/993,946, for example, may be used to deploy the continuous ring intracorneal implant into the pocket.

The continuous ring implant does not have to be folded for insertion. A continuous ring intracorneal implant which is made of a flexible material may be stuffed through the incision without folding. Once fully inserted into the intracomeal pocket, the continuous ring intracorneal implant is straightened out and positioned around the optical zone of the cornea. Preferably, the continuous ring intracorneal implant is positioned remotely from the incision, so that no unnecessary stress is exerted on the incision during healing.

The implant may be implanted by a variety of techniques. Standard forceps may be used to grip and advance the implant into the pocket through the incision as described , or, the forceps may be provided with specialized end effectors, such as that disclosed in co-pending U.S. application Ser. No. 08/993,946, for example, to more positively grip and insert the implant.

The implant may also be inserted into the pocket in a stretched state. The implant may be assembled over features of an insertion tool, such as that disclosed in co-pending U.S. application Ser. No. 08/993,946, for example, adapted to maintain the implant in a stretched state. Once inserted into the corneal pocket, the insert is released from the stretched state and then positioned within the pocket as described above.

Again any stromal debris is removed from the incision area, and the incision area is thoroughly irrigated with balanced salt solution. The tissue edges of the incision are gently approximated to close, and the incision may be closed with one to two interrupted sutures using an ophthalmic suture, preferably 10-0 or 11-0 nylon or equivalent, although it is preferable not to use sutures, staples, glue or any other additional connector or approximation means. The suture depth should be to the level of the stromal pocket. Care should be taken to avoid microperforation by the suture needle. If two sutures are placed, the sutures should trisect the incision line from the superior and inferior aspects of the incision to insure apposition of the anterior edges of the incision.

The anterior incision edges must be opposed to prevent epithelial cells from entering the incision. Care should be taken to ensure that tension across the sutures is evenly applied, however overtightening of the sutures should be avoided as this may induce astigmatism.

Split Rings/Gapped Rings/Circumferential Segments

Initially, the geometric center of the cornea is marked with a blunt instrument (e.g., a Sinskey hook) using an operating microscope for fixation. A zone marker (e.g., an 11 mm zone marker) can be used to aid in locating the center point. A sterile marking pen may be used to enhance the mark. This center mark is used as the reference point throughout the surgical procedure.

Next, the contact surface of an incision and placement marker is marked, using a sterile marking pen, for example. The incision and placement marker is then centered on the center mark created at the geometric center described above, by lining up the reticle of the incision and placement marker with the center mark. The contact surface of the marker is contacted lightly against the cornea, making an inked marking of where the radial incision will be made and, in the case of segments, where the segments will be positioned. A visual verification is made that the marks are at least 1 mm from the limbus in all directions. If the marks are too close to the limbus, re-marking of the geometric center of the cornea is required, to get closer to the actual geometric center.

A pachymetry measurement is made to determine the thickness of the corneal tissue at the incision site. Next, a calibrated, diamond knife is set to 0.430 mm (430μ) or 68% of the intraoperative pachymetry reading taken at the incision site. The diamond should either have an angled cutting edge of 15° or less, or have a rectangular blade of 1 mm width or less.

A radial incision is made by tracing to the outside edge of the incision mark. The incision length may range from about 0.7 to 1.8 mm, and is preferably about 1.2 mm. Special care should be taken to ensure that the incision is kept approximately 1 mm away from the limbus. The incision area is then thoroughly irrigated with balanced salt solution after completing the incision. A Merocel® spear or equivalent is used to remove any loose epithelial cells and excess balanced salt solution from the edges of the incision. The epithelium may be rolled away from the incision edges. The incision is again thoroughly irrigated with balanced salt solution prior to any instrument insertion.

A specialized pocketing tool, such as those described in co-pending U.S. application titled “CORNEAL POCKETING TOOL”, filed on Dec. 18, 1997, is next used to separate the stromal layers at the appropriate depth at the base of the incision. After placing the pocketing tool instrument in the incision, the pocketing tool is rotated to create an intrastromal separation or pocket.

The pocket may be enlarged as desired using a stromal spreader such as is described in co-pending U.S. Application Ser. No. 08/896,792 filed on Jul. 18, 1997 titled “OPTHALMOSURGICAL INSTRUMENTS AND METHODS OF USE”. The tip of the spreader is inserted vertically down into the incision until it contacts the bottom of the incision. A blunt dissection or enlarged pocket is then created on one side of the base of the incision by carefully rotating the blade of the spreader instrument within a single stromal plane. The procedure is then repeated on the other side of the incision base. The resultant pockets should be at the same depth as the incision base, as wide as the full incision length, and extend to the full length of the spreader tip.

Corneal thickness gauges, such as those described in co-pending U.S. patent application Ser. No. 08/676,377, for example, may be used to estimate the depth of both pockets. If the pockets are not deep enough in the corneal stroma, the incision is made slightly deeper with the diamond knife and a second pocket or set of pockets are then created at a deeper level with the pocketing tool and spreader in the manner described above.

The incision and placement marker is next indexed into a vacuum centering guide (VCG) such as those described in Loomas, U.S. Pat. No. 5,403,335 or pending U.S. patent application Ser. No. 08/796,595, for example. The reticle is aligned with the center mark to center the VCG on the center mark. The VCG is then lowered to contact the sclera of the eye while maintaining centration, and then vacuum is slowly applied. Placement of the VCG over the incision and placement marker, together with proper alignment of the marker on both the center mark and the actual incision, ensure that a window in the VCG is centered about the incision site. The vacuum should start in the range of 12-15 inches of Hg. Once a vacuum seal has been established, a confirmation that the VCG is properly placed is made, by checking centration. If the VCG is not properly positioned, the vacuum must be released, and the above procedure regarding the VCG must be repeated. If the VCG is determined to be properly positioned, the vacuum is then slowly increased to 18-20 inches of Hg. It is recommended that the vacuum not exceed 22 inches of Hg. Next, the incision and placement marker is removed from the VCG.

While maintaining the position of the VCG, a counterclockwise (CCW) dissector, such as that described in copending U.S. application Ser. No. 08/676,377, for example, is inserted into the VCG. The dissector body should be rotated until the tip of the dissector blade is adjacent to the incision site. A counterclockwise glide, such as described in U.S. application Ser. No. 08/896,792, for example, is inserted in the incision, at least 1 mm into the pocket and the dissector tip is rotated under the foot of the glide. Counterclockwise rotation of the dissector body allows the dissector tip to enter the pocket underneath the glide. The dissector blade is then advanced approximately 1 mm to 2 mm, then stopped. The glide is removed while leaving the dissector tip in position in the pocket.

While holding the VCG vertically with one hand, the dissector is rotated counterclockwise from the incision to create a stromal channel. Rotation of the dissector in a counterclockwise direction is continued until the support spoke of the dissector blade contacts the incision edge. Then the dissector blade is removed from the channel by rotating the dissector body clockwise until the dissector tip exits the channel. The dissector is then removed from the VCG.

While maintaining the position of the VCG, a clockwise (CW) dissector, such as that described in copending U.S. application Ser. No. 08/676,377, for example, is inserted into the VCG. The dissector body is rotated until the tip of the dissector is adjacent to the incision site. Next, a clockwise (CW) glide, such as described in U.S. application Ser. No. 08/896,792, for example, is inserted at least 1 mm into the opposite pocket and the dissector tip is rotated under the foot of the glide. Clockwise rotation of the dissector body drives the dissector tip into the pocket. The dissector tip should be inserted underneath the glide foot to enter the pocket. The glide blade is next advanced approximately 1 mm to 2 mm, then stopped in its position. The glide is removed while leaving the dissector tip in position in the pocket.

While holding the VCG vertically with one hand, the dissector is rotated clockwise from the incision to create a second stromal channel. The clockwise rotation of the dissector is continued until the support spoke of the dissector blade contacts the incision edge. Then the dissector blade is removed from the channel by rotating the dissector body counterclockwise until the dissector tip exits the channel. The dissector is then removed from the VCG.

If, upon contact of the support spoke of the dissector blade with the incision edge, an abrupt decrease in the resistance has not yet been felt, this generally means that the stromal channels have been formed at different depths and have not met, joined or connected. In this case, a clockwise probe, such as that described in copending U.S. application Ser. No. 08/676,377, for example, is inserted into the clockwise channel, when a split ring is to be implanted. It is not absolutely necessary that the stromal channels be joined for implantation of circumferential segments. If breakthrough occurred, the probe tip will easily rotate past 200° clockwise rotation. Optimally, the probe will rotate past 200° and clearly into the channel created by the counter-clockwise dissector. If, however, the probe does not rotate past 200°, it will be possible to determine which channel, the clockwise or the counter-clockwise channel is the lower channel. Clockwise and counter-clockwise probes, such as those described in copending U.S. application Ser. No. 08/676,377, for example, are inserted into the clockwise and counter-clockwise channels, respectively. The lower channel is determined by observing the position of the probes at the point where their tips overlap. Once this has been determined, the probes are removed from the channels by rotating them clockwise for the counter-clockwise probe and counter-clockwise for the clockwise probe.

If it has been determined that the clockwise channel is the lower one, a clockwise channel connector, such as that described in copending U.S. application Ser. No. 08/676,377, for example, is inserted clockwise into the clockwise channel and rotated clockwise until it is observed to break through into the counter-clockwise channel or until it has rotated about 330°. If the channels are connected, there will have been an abrupt decrease in resistance to continued dissection. If this is the case, the clockwise channel connector is then rotated counter-clockwise and removed from the clockwise channel. If the channels do not meet, a clockwise finish channel connector, such as that described in copending U.S. application Ser. No. 08/676,377, for example, is inserted into the lower, clockwise channel and rotated clockwise until the lower channel connects with the upper channel, or until the finish channel connector tip rotates through to the entry incision. Channel connecting may be determined when an abrupt decrease in resistance to continued dissection is observed. Once the clockwise and counter-clockwise channels have been connected, the finish channel connector is removed.

If it has been determined that the counter-clockwise channel is the lower one, a counter-clockwise channel connector, such as that described in copending U.S. application Ser. No. 08/676,377, for example, is inserted counter-clockwise into the counter-clockwise channel and rotated counter-clockwise until it is observed to breakthrough into the clockwise channel or until it has rotated about 330°. If the channels are connected, there will have been an abrupt decrease in resistance to continued dissection. The counter-clockwise channel connector is then rotated clockwise and removed from the counter-clockwise channel.

If the channels do not meet, a counter-clockwise finish channel connector, such as that described in copending U.S. application Ser. No. 08/676,377, for example, is inserted into the lower, counter-clockwise channel and rotated counter-clockwise until the lower channel connects with the upper channel, such that is there is an abrupt decrease in resistance to continued dissection or until the finish channel connector tip rotates through to the entry incision. Once the clockwise and counter-clockwise channels have been connected, the finish channel connector is removed.

Alternatively, a complete circular interlamellar pathway can be created using a single circular dissector tool, such as described in copending U.S. application Ser. No. 08/993,946, for example. The dissector is inserted and rotated in much the same manner described above with respect to the clockwise and counterclockwise dissectors.

The vacuum is next released and the VCG is removed from the eye. Any stromal debris from the incision site is removed and the incision area is again thoroughly irrigated, using balanced salt solution, prior to insertion of each segment into the stromal channel. Optionally, a small amount of Celluvisc® or an equivalent lubricating agent may be applied to the surface of the cornea, to avoid direct contact of the segments with the epithelium, although this is not preferred.

Each segment, or the ring, is picked up using forceps, such as those described in copending U.S. application Ser. No. 08/896,792, for example. The leading end of each segment, or of the split ring is fed, into the stromal channel from the incision. One segment is rotated clockwise and the second segment is rotated counterclockwise. A ring may be inserted in either a clockwise or counterclockwise manner. The segments have an anterior/posterior orientation. The segment should be placed in the stroma concave side down, such that the cone angle of the segment is most closely matched with the curvature of the cornea.

Using the forceps or a Sinskey Hook, the ring or the segments are manipulated into the desired location within the channel, aligning the outside edge of the segments with the appropriate ink markings left by the incision and placement marker, and the leading ends of the segments are aligned with the appropriate ink markings created by the incision and placement marker.

Again any stromal debris is removed from the incision area, and the incision area is thoroughly irrigated with balanced salt solution. The tissue edges of the incision are gently approximated to close, preferably without the use of any additional connecting or approximation means. Alternatively, the incision may be closed with one to two interrupted sutures using an ophthalmic suture, preferably 10-0 or 11-0 nylon or equivalent. The suture depth should be to the level of the stromal pocket. Care should be taken to avoid microperforation by the suture needle. If two sutures are placed, the sutures should trisect the incision line from the superior and inferior aspects of the incision to insure apposition of the anterior edges of the incision.

The anterior incision edges must be opposed to prevent epithelial cells from entering the incision. Care should be taken to ensure that tension across the sutures is evenly applied, however overtightening of the sutures should be avoided as this may induce astigmatism.

Radial Implants/Composite Radial Implants

Initially, a center mark is made at the geometric center of the cornea using a blunt instrument and an operating microscope or other comparable technique that accurately marks the center of the cornea. Next, a corneal marker, such as that described in copending U.S. application Ser. No. 08/993,696, filed Dec. 18, 1997 and entitled “Radial Intrastromal Corneal Implant and a Method of Insertion, is aligned with the center mark and pressed onto the cornea, marking the cornea with a incision marks, which are preferably symmetrically spaced about a circumferential locus of the cornea. Separate marks are made for each of the incisions to be made, respectively.

An incision (preferably a circumferential incision) is next made, at each incision mark, into the cornea, thereby cutting through some but not all of the stroma. A small “starter” pocket is formed at the base of each incision.

The incision is made using any appropriate surgical or diamond blade typically having a footplate on one or both sides of the blade to control the overall depth of the incision. Once the incision has been made, pocketing between corneal layers may be accomplished using a suitable instrument, such as a dissector or spreader (e.g., as described in U.S. application Ser. No. 08/896,792).

A specialized pocketing tool (such as described in co-pending U.S. application titled “CORNEAL POCKETING TOOL”, filed on Dec. 18, 1997 (attorney docket no. 25169-20053.00), for example) may also be used to separate the stromal layers at the appropriate depth at the base of the incision. Once in place in the incision, the pocketing tool can be rotated to create an intrastromal separation or pocket. This small starter pocket may be enlarged as desired using a stromal spreader.

Once the pockets have been formed, the radial inserts are then placed at the back end of each respective pocket as described in copending U.S. application Ser. No. 08/993,696. The tissue edges of the incisions are gently approximated to close, preferably without the use of any additional connecting or approximation means. Alternatively, the incision may be closed with the additional use of sutures, glue, staples or by electrosurgical welding. When using sutures, the incisions may be closed with one to two interrupted sutures using an ophthalmic suture, preferably 10-0 or 11-0 nylon or equivalent. The suture depth should be to the level of the stromal pocket. Care should be taken to avoid microperforation by the suture needle. If two sutures are placed, the sutures should trisect the incision line from the superior and inferior aspects of the incision to insure apposition of the anterior edges of the incision.

The anterior incision edges must be opposed to prevent epithelial cells from entering the incision. Care should be taken to ensure that tension across the sutures is evenly applied, however overtightening of the sutures should be avoided as this may induce astigmatism.

Alternative to the incisions described above, a small radial incision and pocket may be formed for placement of each insert. The incisions are within the cornea. In another alternative, an incision outside of the cornea, within the limbus of the eye, may be made for each insert, respectively, through which the respective insert is placed into the cornea.

Alternative Methods for Radial Implants/Composite Radial Implants

(1) Initially, a center mark is made at the geometric center of the cornea using a blunt instrument and an operating microscope or other comparable technique that accurately marks the center of the cornea. Next, a corneal marker, such as that described in copending U.S. application Ser. No. 08/993,696, for example, is aligned with the center mark and pressed onto the cornea, marking the cornea with an incision mark, with clockwise and counter-clockwise circumferential channel marks, and with radial pocket marks.

An incision is made into the cornea at the incision mark, thereby cutting through some but not all of the stroma. Next, a vacuum centering guide ( such as described in U.S. Pat. No. 5,403,335 or pending U.S. patent application Ser. No. 08/796,595, for example) is positioned on the cornea using the centering mark on the cornea, and a vacuum of approximately 10-27 in. Hg is drawn to hold the vacuum centering guide on the eye. Small “starter” pockets are formed at the base of the incision perpendicular to the incision and in the direction that the circumferential channels are to be formed.

The incision is made using any appropriate surgical or diamond blade typically having a footplate on one or both sides of the blade to control the overall depth of the incision. Once the incision has been made, pocketing between corneal layers may be accomplished using a suitable instrument, such as a dissector or spreader (e.g., as described in U.S. application Ser. No. 08/896,792).

A specialized pocketing tool may also be used to separate the stromal layers at the appropriate depth at the base of the incision. Once in place in the incision, the pocketing tool can be rotated to create an intrastromal separation or pocket. This small starter pocket may be enlarged as desired using a stromal spreader.

Once the initial separation or pocket has been created in the manner described above, a clockwise dissector blade, such as described in copending U.S. application Ser. No. 08/676,377, for example, is inserted into the vacuum centering guide, and a blunt-tipped instrument (e.g., glide) is inserted into one of the small “starter” pockets. The corneal tissue is lifted to allow insertion of the tip of the dissector blade into the starter pocket. Next, the dissector blade is rotated to separate the stroma and form a clockwise circumferential channel between stroma. The clockwise dissector blade is removed and the procedure is repeated using the counter-clockwise dissector blade to form a counter-clockwise circumferential channel. Separate, unjoined circumferential channels of any arc length or a continuous 360° channel can be formed using this method.

A radial pocket is formed by inserting a radial pocket-forming instrument (such as that described in copending U.S. application Ser. No. 08/993,696, for example) through the single incision and into the circumferential channel a sufficient distance that a tissue separator (such as a blade) on the instrument either is under one of the radial pocket marks on the cornea that crosses the circumferential channel or is adjacent to one of the radial pocket marks that ends at or near the circumferential channel. The radial pocket-forming instrument is then rotated or translated laterally so that the blade engages the sidewall of the circumferential channel and separates stroma to form a radial pocket connected to the circumferential channel and located beneath the radial pocket mark. The length, width, and shape of the pocket are determined by the size and shape of the blade. The radial pocket-forming instrument is next rotated or translated in the opposite direction to remove the blade from the radial pocket and is repositioned adjacent to another radial pocket mark to form another radial pocket. When all radial pockets have been formed, the radial pocket-forming instrument is withdrawn from the circumferential channel. The cornea is thus prepared to receive an intracorneal insert.

A radial insert is inserted into the circumferential channel through the incision in the cornea. An instrument may be used to push or pull the radial insert to a position adjacent to a radial pocket. The same instrument or another positioning instrument (such as described in copending U.S. application Ser. No. 08/993,696, for example) is then used to maneuver one end of the radial insert into the radial pocket and to maneuver the other end of the radial insert against the sidewall of the circumferential channel that is opposite to the radial pocket. If more than one radial pocket is connected to a circumferential channel, the first radial insert is placed into the radial pocket that is located farthest from the single incision into the cornea. The next radial insert is placed into the radial pocket that is second farthest from the single incision, and so forth.

Short circumferential inserts may be inserted between adjacent radial inserts, if desired. For example, a radial insert may be inserted as discussed above into the farthest radial pocket from the single incision, and next a circumferential insert may be placed into the circumferential channel so that the circumferential insert abuts the radial insert. The circumferential insert is shorter than or the same length as the distance in the circumferential channel between adjacent radial pockets. Another radial insert is then placed into the next farthest radial pocket and then a circumferential insert, and so forth. A single circumferential insert may be placed into the circumferential channel to both hold the radial inserts in their pockets and to further reshape the patient's cornea. The number of inserts and the size and shape of each circumferential insert and radial insert are determined by the amount of reshaping of the cornea that is needed to provide a spherically-shaped cornea in the patient's eye.

(2) A small meridional incision is made in the outer periphery of the anterior surface of the cornea. The slit may be circumferential if so desired. Next, a dissector, such as is shown in U.S. Pat. No. 5,403,335 to Loomas et al, issued Apr. 4, 1995, is then introduced into the initial incision and rotated to form the circumferential interlamellar tunnel.

Next, a first radial insert is introduced through the initial slit and into the intrastromal channel. The radial insert is then rotated into the desired meridional position within the channel, for example, at the 12:00 position. Other inserts are introduced in the same fashion. The initial opening is then closed by use of a suture, glue, staple, or by electrosurgical welding. The radial inserts may be introduced into the channel by way of the incision in any appropriate manner. For example, the insert may be grasped and manipulated through the incision and into the channel using standard micro-forceps. Preferably, the forceps are constructed with tip ends having enhanced gripping features to positively hold the insert against sliding or rotation relative to the tip ends. Such features may include recesses or indentations adapted to receive a portion of the insert, protuberances, gripping teeth, or other such features constructed to positively hold the insert. The exact configuration depends upon the shape of the insert and the preference of the surgeon.

Alternatively an introducer apparatus capable of holding and controllably inserting one or more inserts into the channel may be used. Suitable instruments for placing an insert into an intracorneal channel can be found in “CORNEAL IMPLANT INTRODUCER AND METHOD OF USE”, filed on Dec. 18, 1997.

Turning now to the figures, FIG. 1 shows an eye of a patient having an intracorneal ring implant 20 implanted within the cornea C of the eye. The cornea C of an adult human eye typically has a diameter of approximately 12 mm. The cornea C has the general shape of a circular section of a sphere. The perimeter of the cornea C, called the limbus L, separates the clear cornea C from the opaque white sclera S of the eye. Through the cornea C can be seen the iris I of the eye which surrounds the pupil P. The intracomeal ring implant 20 is surgically implanted within the cornea C by insertion through an incision 22 in the anterior surface of the cornea C into a channel or pocket formed within the stromal layers of the cornea C, as described above.

The intracorneal ring implant 20, according to the present invention, has a geometry of a ring-shaped section of a cone, approximating a section of a sphere. The intracorneal ring implant 20 has a centroid diameter of approximately 6.8 to 8.1 mm, more preferably about 7.0 to 7.8 mm and most preferably about 7.5 mm. The centroid diameter is defined as the distance across the ring when measured from the centroid points of the cross-section of the material forming the ring, as shown by reference d in FIG. 3. When the implant comprises segments 120, as shown for example, in FIG. 20, a centroid diameter, measuring the distance between the centroids of the segments 120, is likewise approximately 6.8 to 8.1 mm, more preferably about 7.0 to 7.8 mm and most preferably about 7.5 mm. The intracorneal implant, whether in the form of a ring, split ring, gapped ring or segments, is implanted into the cornea C just outside of the optical zone, which is approximately the central third of the cornea. The intracorneal implant has at least one region of modified elasticity. In the exemplary embodiment of the intracorneal ring implant 20 shown in FIG. 1, there are four sector-shaped regions of varying elasticity. The first region 24 and the third region 28 have a relatively high bulk or apparent (or both) modulus of elasticity. The second region 26 and the fourth region 30 have a relatively low bulk or apparent (or both) modulus of elasticity, compared to regions 24 and 28. The four regions combined subtend a total angle of 360 degrees to form a complete circular ring. The intracorneal ring implant 20 may optionally have a split 32 somewhere along the ring to facilitate insertion of the implant 20. Alternatively, the ring may have a gap so as not to subtend a full 360 degrees.

As shown in FIG. 20, the implant may comprise segments 120, which when implanted, follow the same contour as that of the ring shown in FIG. 1. In this embodiment, each segment 120 has three regions of varying elasticity 122, 124, 126. However, the invention is not to be limited to three regions, as each segment could have only two regions or more than three regions of varying elasticity. The regions 122 and 126 in FIG. 20 have a relatively high bulk or apparent (or both) modulus of elasticity. The regions 124 have a relatively low bulk or apparent (or both) modulus of elasticity.

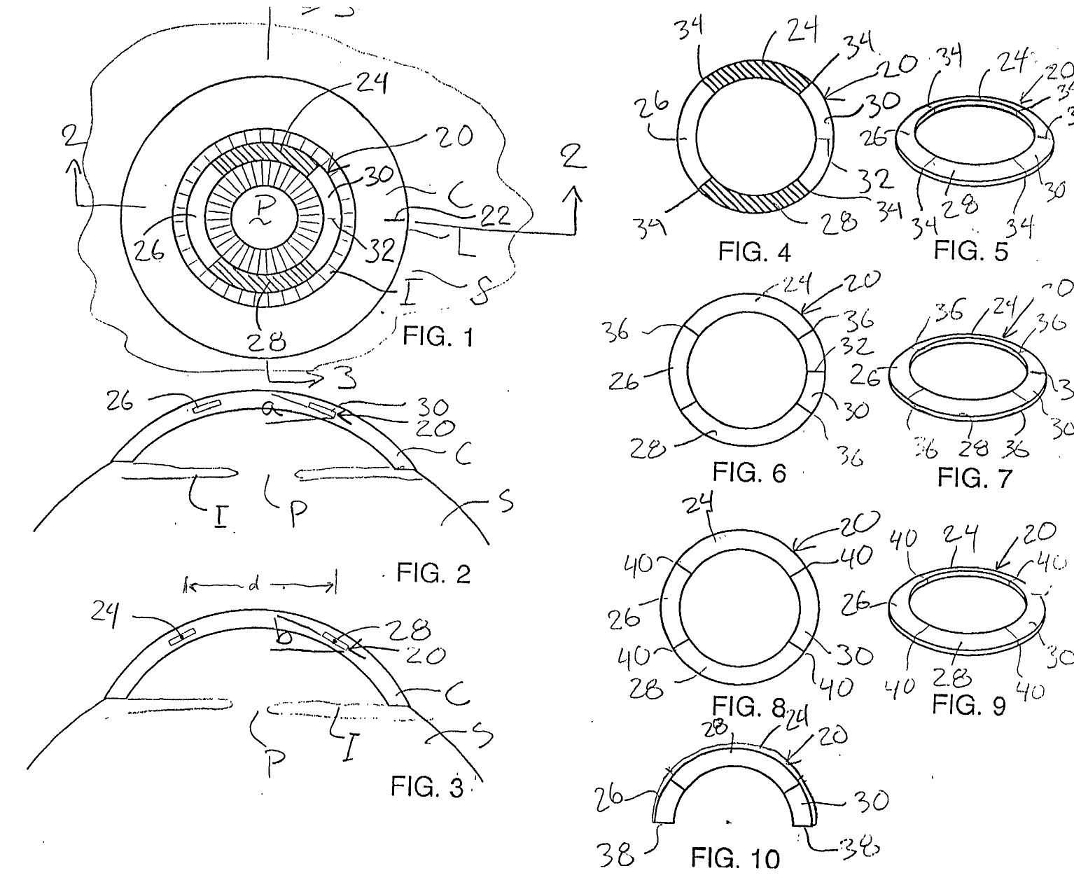

FIG. 2 is a cross section of the cornea C and intracorneal ring implant 20 of FIG. 1, taken along section line 2-2. The second region 26 and the fourth region 30 of the implant 20 are seen in cross section within the cornea C. Because of the relatively low bulk and/or apparent modulus of elasticity of the second region 26 and the fourth region 30, these regions of the ring are flexible and conform closely to the native curvature of the cornea C, substantially aligning with the lamellae 12 as shown in FIG. 26, with a relatively matched cone angle, e.g. about 28° to 32° for a ring having a centroid diameter of about 7.5 mm. As such, the second region 26 and the fourth region 30 have a different effect on the curvature and therefore the refractive power of the cornea C along the optical meridians which pass through these regions. A more complete discussion of the cone angle is described in copending U.S. application Ser. No. 08/827,624, filed on Apr. 10, 1997, which is herein incorporated by reference in its entirety.

FIG. 3 is a cross section of the cornea C and intracorneal ring implant 20 of FIG. 1, taken along section line 3-3. The first region 24 and the third region 28 of the implant 20 are seen in cross section within the cornea C. Because of the relatively high bulk and/or apparent modulus of elasticity of the first region 24 and the third region 28, these regions of the ring are stiffer and maintain a mismatched cone angle b. The degree of mismatch is highly dependent upon the geometry of the ring and generally ranges from about 2° to 20° smaller or larger than the matching angle, which changes the curvature in the sectors of the cornea C affected by these regions differently than in the other regions and less than the matching angle (FIG. 28), respectively), thereby exerting a varying curvature effect on the cornea (variable modulus effect).

FIGS. 27 and 28 show effects on the cornea by a mismatched cone angle which is greater than the matching angle (FIG. 27) and less than the matching cone angle (FIG. 28), respectively. In other words, the respective cone angles do not match the inner lamellar architecture of the cornea. The phantom lines shown in these figures correspond to the outer lines of the cornea section of FIG. 26 and thus provide a reference for the corneal configuration changes caused by the respective mismatching cone angles. Referring particularly to FIG. 27, regions or zones 24 and 28 are shown with cone angles greater than those shown in FIG. 26. The larger cone angle twists adjacent portions of the cornea outward and flattens the central region of the cornea between regions 24 and 28 as shown in the drawing.

In contrast, FIG. 28 shows the effect when regions 24 and 28 have mismatching cone angles which are less than the matching angle, i.e., than that shown in FIG. 26. This smaller cone angle effects a steepening of the corneal surface C as shown in the drawing.

The changed curvature in these sectors of the cornea C changes the refractive power of the cornea C along the optical meridians which pass through the first region 24 and the third region 28 of the implant 20. Thus, the employment of a variable modulus ring or variable modulus segments effects different regions of the cornea differently.

By varying the refractive power of the cornea C along the optical meridians which pass through the first region 24 and the third region 28 of the implant 20 to a greater degree than along the optical meridians which pass through the second region 26 and the fourth region 30 of the implant 20, the astigmatism due to irregularities in the curvature of the cornea can be corrected so that light rays entering the eye parallel to the optical axis are properly focused at a point on the retina.

The intracorneal ring implant 20 can also be made to correct for myopia or hyperopia which is superimposed on an astigmatism. For correction of myopia, the intracorneal ring implant 20 can be made with sufficient bulk around the entire ring (or segments) so that it flattens out the curvature of the cornea, reducing its refractive power in all sectors, while the varying cone angle due to the regions of varying effective elastic modulus correct the astigmatism. In using the bulk approach, the modulus of the material is relatively unimportant and the material need only have enough compressive strength to hold its shape against the compressive forces of the cornea. Thus, the material used in this approach may be very flexible.

Alternatively, myopia can be corrected by making the intracorneal ring implant 20 with a cone angle in all regions which is less than the native tangent angle of the cornea without the implant 20. The lower cone angle tends to flatten out the curvature of cornea, reducing its refractive power in all sectors, while the varying cone angles due to the regions of varying effective elastic modulus correct the astigmatism. For correction of hyperopia, the intracorneal ring implant 20 can be made with a cone angle in all regions which is greater than the native tangent angle of the cornea without the implant 20. The higher cone angle tends to increase the curvature of the cornea, increasing its refractive power in all sectors, while the varying cone angles due to the regions of varying effective elastic modulus correct the astigmatism.

There are a number of ways of achieving the regions of varying effective elastic modulus in the intracorneal implant which apply equally to ring, modified ring (split or gapped) and segment implants. FIG. 4 shows a plan view of an embodiment of the intracorneal ring implant 20 having a first region 24, a second region 26, a third region 28 and a fourth region 30. FIG. 5 shows this same embodiment in a perspective view. By way of example only, each region in this embodiment is illustrated as subtending an angle of approximately 90 degrees. In other embodiments, the regions may subtend lesser angles, as low as around 30° to 45°. Regions subtending an angle of less than about 30° tend to be less than completely effective in achieving the varied modulus effects described above. Of course, regions which subtend an angle greater than 90° may be effectively employed if so desired. The first region 24 and the third region 28 are made of a biocompatible material with a relatively high bulk and/or apparent modulus of elasticity. The second region 26 and the fourth region 30 are made of a biocompatible material with a relatively low effective modulus of elasticity. For example, the high bulk and/or apparent modulus material may have a bulk and/or apparent modulus of elasticity in the range of about 10,000 to 350,000 psi, and the low bulk and/or apparent modulus material may have a bulk and/or apparent modulus of elasticity in the range of about 1 to 90,000 psi.

Suitable materials for making the intracomeal implants include physiologically acceptable polymers such as polystyrene, acetals, polyesters, polycarbonates, polyurethanes, acrylics such as PMMA, physiologically compatible elastomers, crosslinked polymeric gels such as polyhydroxyethylmethacrylate (Poly-HEMA) or polyvinylpyrrolidone (PVP), polyethylene oxide, or polyacrylates, polyacrylic acid and its derivatives, their copolymers and interpolymers, and the like as well as semi-synthetic polymers such as crosslinked dextran, crosslinked heparin or hyaluronic acid. A number of polymeric layers may be employed to include a soft or hydratable polymer on their outer surface. Partially hydrated or filly hydrated hydrophilic polymers are typically slippery and consequently may contribute to the ease with which the implant may be introduced.

The implant may be lubricated with suitable ocular lubricants such as hyaluronic acid, methylethyl cellulose, dextran solutions, glycerine solutions, polysaccharides, or oligosaccharides upon its introduction to help with the insertion particularly if one wishes to insert an implant made of hydrophilic polymers without prior hydration.

Low bulk or apparent modulus polymers used in this invention are often absorbent, particularly if they are hydratable, and may be infused with a drug or biologic agent which may be slowly released from the device after implantation of the intrastromal segment. For instance, the low bulk or apparent modulus polymer may be loaded with a drug such as dexamethasone to reduce acute inflammatory response to implanting the device. This drug helps to prevent undesirable scarring or vascular in growth toward the intrastromal segment. Similarly, heparin, corticosteroids, antimitotics, antifibrotics, antiinflammatories, anti-scar-forming, anti-adhesion, and antiangiogenesis factors (such as nicotine adenine dinucleotide (NAD⁺)) may be included to reduce or prevent angiogenesis and inflammation.

Clearly, there are a variety of other drugs suitable for inclusion in the intrastromal segment. The choice will depend upon the use to which the drugs are put.

The materials for each region are chosen from the candidate materials for the appropriate modulus of elasticity. The materials for each region are preferably chosen for chemical compatibility with one another to facilitate manufacturing of the implant 20. For example, the first region 24 and the third region 28 could be made of a high molecular weight polymer for rigidity, while the second region 26 and the fourth region 30 could be made of a lower molecular weight version of the same polymer, or a different polymer for flexibility.

Alternatively, the first region 24 and the third region 28 could be made of a relatively rigid, highly crosslinked hydrogel, while the second region 26 and the fourth region 30 could be made of a relatively flexible, less crosslinked hydrogel. Using compatible materials or materials from the same family for the different regions allows the regions to be directly fused to one another without the necessity of additional adhesives. However, different polymers may also be employed for different moduli but it is preferable that if different polymers are selected, they are selected so as to be bondable with one another. In an exemplary embodiment, the regions may be joined to one another by radially oriented buttjoints 34. Consequently, there is a relatively abrupt change in stiffness at the joints between the regions.

The intracorneal ring implant 20 in this embodiment has a split 32 in the ring to facilitate insertion of the implant 20 into the cornea by a method such as described above, for example. Preferably, the split 32 in the ring is made in the more flexible second region 26 or fourth region 30 of the implant 20, as splitting the first region 24 or the third region 28 would be counter to the desire to make these regions relatively rigid. In addition, placing the split 32 in one of the flexible regions will lessen the stress on the corneal tissue situated above the split 32 after surgical implantation.

Alternative to butt joints or other forms of abrupt modulus transition, a more gradual transition may be employed. For example, a tapered, overlapping joint 34′ such as that shown in FIG. 21 may be used to join sections of varying modulus 26′and 24′. Additionally, FIG. 6 shows a plan view and FIG. 7 shows a perspective view of an alternate version of the intracorneal ring implant 20. In this version, the first region 24, second region 26, third region 28 and fourth region 30 are joined to one another by lap joints 36, which results in a gradual transition of stiffness from more rigid regions to the more flexible regions. FIG. 21 is a side view of an implant 20 in which regions 72 and 74, which have diverse bulk and/or apparent moduli of elasticity, are joined by a gradually tapering joint 70 to give a continuously varying stiffness along the region defined by the joint 70. Whether the abrupt stiffness transition of the butt-joined version of FIGS. 4 and 5 or the more gradual stiffness transition of the lap-joined version of FIGS. 6 and 7 or FIG. 21 is more appropriate depends on the exact nature of the astigmatism which is to be corrected.

FIG. 8 shows a plan view and FIG. 9 shows a perspective view of a continuous ring version of the intracomeal ring implant 20 without a split in the ring. The first region 24, second region 26, third region 28 and fourth region 30 form a continuous circle joined together by joints 40, which may be buttjoints, lap joints or any suitable joint. This embodiment of the intracorneal ring implant 20 is intended to be folded along a line 38 which passes through the more flexible second region 26 and fourth region 30 as shown in FIG. 10 for implantation into a pocket formed within the cornea by the method described in U.S. patent application Ser. No. 08/993,946 filed Jul. 18, 1997 for “Corneal Implant Methods and Pliable Implant Therefor”.

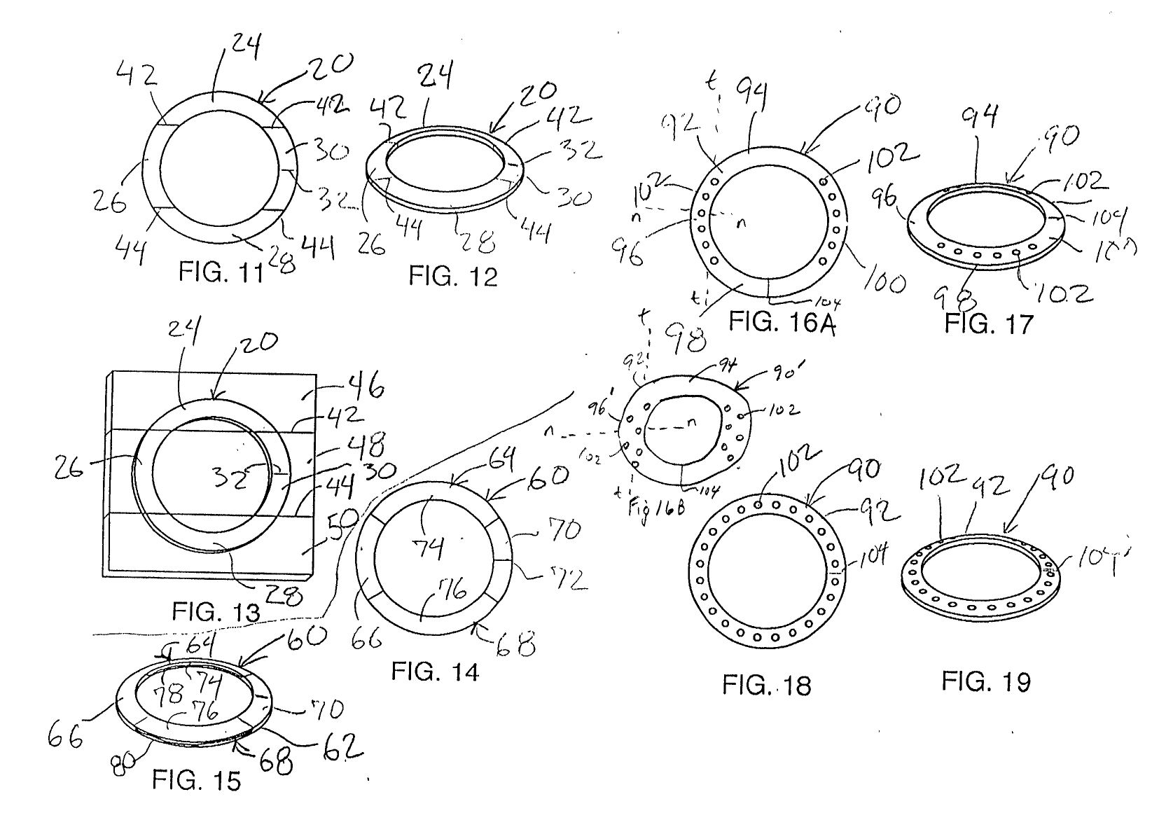

FIG. 11 shows a plan view and FIG. 12 shows a perspective view of a version of the intracorneal ring implant 20 where the relatively rigid first region 24 and third region 28 are joined to the relatively flexible second region 26 and fourth region 30 by joints that are oriented along two pairs of parallel lines 42, 44. These joints 42, 44 may be formed by any of the joints described above, as desired. This embodiment facilitates a method of high volume production with high consistency and repeatability. This version of the intracorneal ring implant 20 can be manufactured from a continuously coextruded ribbon 52 with three bands of material 46, 48, 50 made from the high bulk and/or apparent elastic modulus, rigid material and the lower bulk and/or apparent elastic modulus, flexible material, as illustrated in FIG. 13. An example of a band described by both bulk and apparent modulus would be one that has both a homogeneous material, as well as a strip, strand or other subsection formed from a modified or composite material. The coextruded ribbon 52 can be made with two bands of flexible material 46, 50, which form the flexible regions 24, 28 of the implant 20, surrounding a single band of rigid material 48, which forms the relatively rigid regions 26, 30 of the implant 20, as shown in FIG. 13. Alternatively, the coextruded ribbon 52 can be made with two bands of rigid material surrounding a single band of flexible material. The choice will depend on the desired orientation of the joints 42, 44 between the different regions in the finished implant 20. The coextruded ribbon 52 is punched out to create a ring, and then thermoformed to create the desired cone angle for the implant 20. If desired a split 32 is cut in the ring, preferably in one of the flexible regions 24, 28. Of course, a gapped ring, as well as segments may be formed similarly.

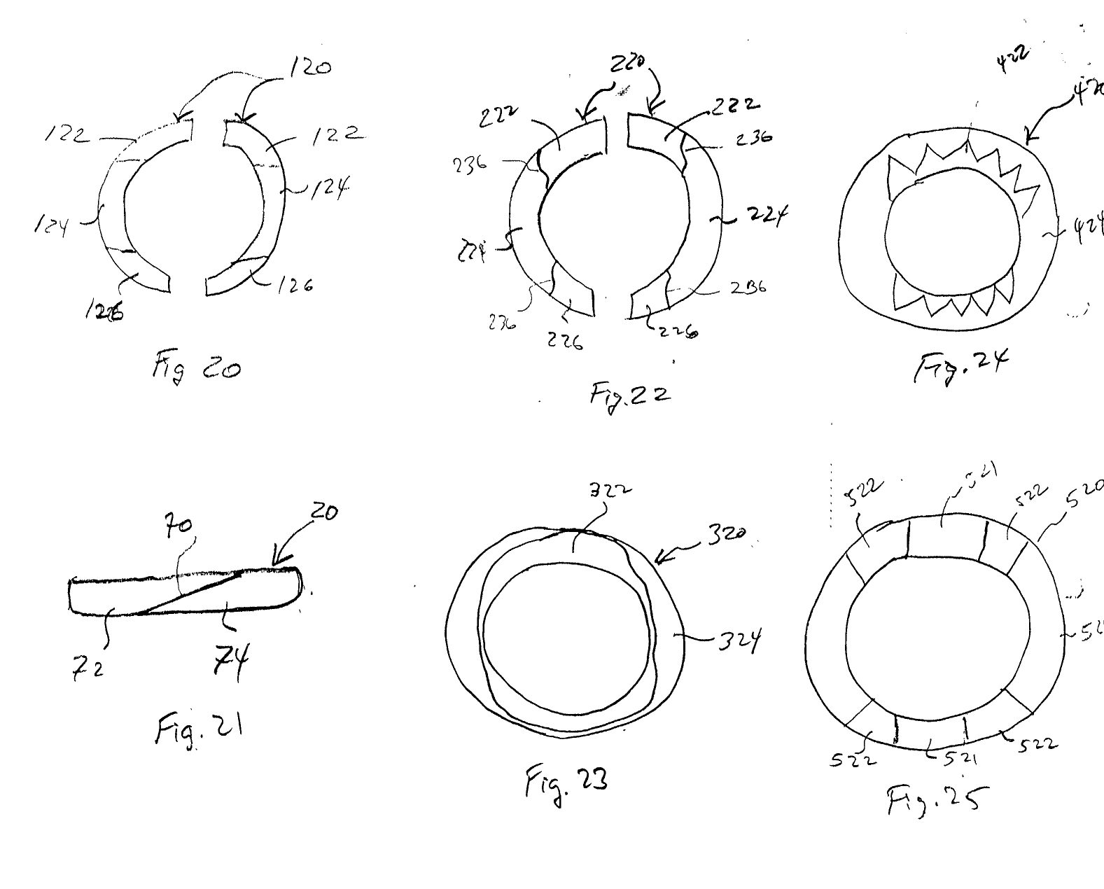

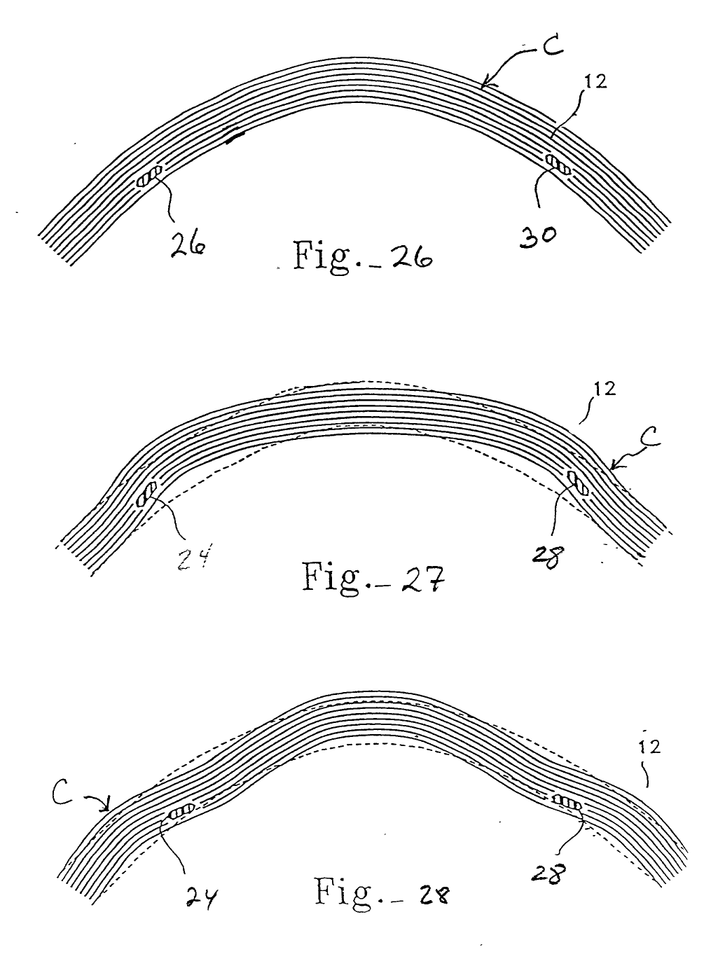

Preferably, however, the implants will be formed by co-casting the varying modulus materials into the desired shape of a ring, split ring, gapped ring or segments having the desired cone angles. Any of the joints previously described can be accomplished by co-casting. Additionally, there is almost no limit to the configurations of joints as well as modifications of bulk or apparent modulus that may be accomplished in the respective sections by co-casting. For example, FIG. 22 shows irregularly, but symmetrically formed joints 236 at the intersections of the co-casted portions 222, 224 and 226, respectively. An advantage of this ability to form the joints according to nearly any conceivable pattern, is that the transition between segments can then be aligned according to the measurements taken for the particular astigmatism to be treated, according to various stress contours, etc.

Further, the portions of the segments need not be radially separated, but may be formed according to other patterns by co-casting, depending upon how the designer intends the effective modulus to change along each segment/ring. For example, FIG. 23 shows an example where portions 322 and 324 are co-casted to form a ring. Portion 324 has a higher bulk and/or apparent modulus of elasticity than portion 322. Portion 324 is more substantial in the 3 o'clock and 9 o'clock positions and portion 322 is more substantial in the 6 o'clock and 12 o'clock positions, respectively, in the figure. Thus, the portions in the 6 and 12 o'clock positions are more flexible than the portions in the 3 and 9 o'clock positions, with the effective moduli between the portions gradually varying from high to low and vice versa. The patterns for the sections are also almost limitless. Additionally, the co-casted portions can be further treated with radiation or chemical treatments to form covalent linkages between polymers of different sections.

Another alternative is to form the implants using an irradiation process, to either increase the modulus (stiffen) of selected sections by cross-linking or to decrease the modulus (soften) of selected sections by radiation weakening the material through the breaking of some existing bonds. In each case, the nonselected portions are provided with a mask to prevent exposure to the radiation process.

An additional technique for varying the bulk or apparent modulus of elasticity of select portions of an implant is illustrated in FIG. 24. Using a photomask technique, selected portions 422 of the implant 420 may be exposed to various forms of radiation, chemical treatments, etc. to either strengthen or weaken the exposed areas. For example, areas 422 might be exposed to radiation that cross-links the implant material to strengthen or increase its bulk or apparent modulus of elasticity. On the other hand, the areas 422 could be exposed to a radiation or chemical that would partially degrade or otherwise weaken the material to reduce the bulk or apparent modulus of elasticity of sections 422. By these techniques a limitless arrangement of portions can be formed.

Multiple masking, or a single masking with varying degrees of density allowing various degrees of penetration by a radiation to be used, may be employed to form gradations of variation in bulk or apparent modulus of elasticity. For example, FIG. 25 shows an implant in which a first mask was applied to all portions of the implant 520 except for portions 521. Portions 521 were then irradiated to weaken them (reduce the bulk or apparent modulus of elasticity). Next, the first mask was removed and a second mask was applied to cover all parts of the implant except for portions 521 and 522 (alternatively only the portions of the first mask covering portions 522 could be removed, while leaving the remainder of the first mask in place). Another irradiation step is then applied to the exposed portions 521 and 522. After removal of the second mask, the resulting implant 520 has portions 521 with a low bulk or apparent modulus of elasticity, portions 524 with a high bulk or apparent modulus of elasticity, and transition portions 522 which has a lower bulk or apparent modulus of elasticity than portions 524, but a higher bulk or apparent modulus of elasticity than portions 521. As with the single masking techniques, an unlimited number of patterns can be applied to the implant using this technique.

Other treatments for variation of the bulk and/or apparent modulus of elasticity of select portions of an implant include the application of solvents, plasticizers and the like to the portions to be modified.

In additional embodiments of the present invention, described below, the stiffness of the implant portions may be varied by the use of composite constructions or by various additional treatments of the portions. For example, composite sections can be formed by adding chopped fibers, particulate such as TiO₂ and the like, or other fillers to the polymeric matrix. Additionally, many other physical modifications of the sections can be performed to vary the elasticity. In these versions, the stiffness of each portion can be described as depending on an effective elastic modulus, which is determined by the bulk properties of the material(s) in the portion, and upon the dimensions of each. The following examples illustrate this principle.

FIG. 14 shows a plan view and FIG. 15 shows a perspective view of a version of an intracorneal ring implant 60 with two regions 64, 68 made of a layered composite. This embodiment of the intracorneal ring implant 60 is made with a ring-shaped implant body 62 that is molded of a first component material. Sector-shaped inserts 74, 76 of a second component material are joined to the ring-shaped implant body 62 to form the four regions 64, 66, 68, 70 of the implant 60. In the example illustrated the ring-shaped body 62 is molded of a flexible, low modulus material with two depressions or thinned regions 78, 80 in the ring 62 that are filled with inserts 74, 76 molded of a relatively rigid, higher modulus material. The inserts 74, 76 may be joined to the ring-shaped body 62 , for example, by insert molding, heat fusing, insert molding, co-casting , adhesive, etc. The stiffness of the flexible second region 66 and fourth region 70 are determined by the outer dimensions of the ring-shaped body 62 in these regions and the elastic modulus of the more flexible material. The stiffness of the relatively rigid first region 64 and third region 68 can be calculated from the stiffness contributions of the thinned sections 78, 80 of the ring 62 and the sector-shaped inserts 74, 76. Alternatively, the stiffness of first region 64 and third region 68 can be thought of in terms of the outer dimensions of the implant 60 within these regions and the effective elastic modulus of the composite of the rigid material and the flexible material. For the geometry shown, the effective modulus will be approximately equal to the volumetric average of the elastic modulus of the rigid material and the elastic modulus of the flexible material. Optionally, this embodiment may also have a split 72 in the ring, preferably in one of the flexible regions as shown. Additionally, a gapped ring as well as segments may be formed as layered composites according to the principles just discussed.

Alternatively, the ring-shaped body 62 of the intracorneal ring implant 60 may be molded of a relatively rigid, higher modulus material with two depressions in the ring that are filled with inserts molded of a flexible, low modulus material. In this case, the stiffness of the flexible second region 66 and fourth region 70 is determined by the outer dimensions of the implant 60 and the effective elastic modulus of the composite of the rigid material and the flexible material. Other arrangements of composite materials or stiffness-modifying inserts may be used to create the regions of varying elastic modulus within the implant 60. Again, these techniques are not limited to ring implants, but apply equally to split rings, gapped rings and segments.

FIG. 16A shows a plan view and FIG. 17 shows a perspective view of a version of the intracorneal ring implant 90 where the varied stiffness of the different regions is created by changing the internal geometry of the implant 90 within the regions. Like the previously described embodiments, this embodiment of the intracorneal ring implant 90 may be made as a split ring 104 or a continuous ring, gapped ring or segments, depending on the chosen surgical technique for implantation. The implant 90 has a ring-shaped body 92 made of a homogeneous material by any suitable process, such as casting, insert molding, machining, die cutting thernoforning, etc. In order to vary the stiffness of the ring-shaped body 92, the second region 96 and the fourth region 100 of the ring are pierced with holes 102, which render these regions more flexible than the first region 94 and the third region 98. The holes 102 may be molded into the ring-shaped body 92 or they may be created by post processing, such as by drilling, punching, laser cutting, etc. Typically, the second region 96 and the fourth region 100 will each have an array of multiple holes 102. The size and the number of the holes 102 will determine the stiffness of the region, which may be expressed in terms of an effective modulus of elasticity. The arrays of holes 102 may be arranged to create either an abrupt transition in stiffness or a gradual transition in stiffness between the regions by varying the size and/or the spacing of the holes within the array. The holes 102 may pass all the way through the ring-shaped body 92 or they may pass only part way through. If desired, the holes 102 may be filled with a material with a lower modulus of elasticity to create a composite structure with a lower effective elastic modulus than the homogenous material of the first region 94 and the third region 98. Alternatively, other equivalent features may be used to alter the stiffness of the second region 94 and the fourth region 100, such as grooves, dimples, voids, gas bubbles, tortuous cavities, low modulus inclusions within the material, or exposure to radiation or other energy sources which modify the material properties of the selected regions. Further, the ringshaped body 92 may be made non-homogeneous, e.g. having one or more composite sections for additional tailoring of the elasticity characteristics of the ring.

The same principles illustrated in FIG. 16A may be used to modify the stiffniess of the entire ring-shaped body of the intracorneal ring implant 90, as shown in the plan and perspective view of FIG. 18 and FIG. 19, respectively. The implant 90 has a ring-shaped body 92 made of a homogeneous material. The intracorneal ring implant 90 may be made as a split ring 104 or as a continuous ring. The ring-shaped body 92 of the implant 90 is pierced with an array of multiple holes 102 or equivalent features to render the ring 92 more flexible. The holes 102 may be molded into the ring-shaped body or they may be created by post processing, such as by drilling, punching, laser cutting, etc. The size, number and spacing of the holes 102 will determine the stiffness of the ring-shaped body 92, which may be expressed in terms of an effective modulus of elasticity. Although the holes in FIGS. 18 and 19 appear to be equally spaced and sized around the entire circumference of the ring-shaped body 92, the spacing, number and size of the holes can be varied around the circumference to create varying zones of different elastic moduli circumferentially about the ring-shaped body 92. The holes 102 may pass all the way through the ring-shaped body 92 or they may pass only part way through. If desired, the holes 102 may be filled with a material with a lower modulus of elasticity to create a composite structure with a lower effective elastic modulus. The modified stiffness or flexibility of the ring-shaped body 92 will affect the cone angle that the implant can maintain within the cornea and therefore the degree of refractive correction created. By proper choice of the stiffness, cone angle and the bulk of the ring-shaped body 92, this embodiment of the implant 90 can be used for correction of myopia or hyperopia without astigmatism. Further, the ring-shaped body 92 may be made non-homogeneous, e.g. having one or more composite sections for additional tailoring of the elasticity characteristics of the ring.

FIG. 16B illustrate some example of the variety of alternative placements and spacings of the holes 102 that are available to achieve varying flexibility characteristics. As can be seen the holes 102 are less aligned an more evenly distributed through the thickness of the ring 90′ as compared to holes 102 in ring 90 shown in FIG. 16A. As a result, the ring section 96 is more flexible about an axis t which is tangent to the ring. The ring section 96′ maintains a flexibility about an axis n normal to the tangent axis t which is approximately the same as the flexibility of ring section 96 about a normal axis n. However, the ring section 96′ is somewhat stiffer about the tangential axis t as compared to the ring section 96. Because there is no limit to the placement of the holes 102 in three dimensions within the confines of the ring, there are an infinite number of alternative arrangements that can be tailored to control the stiffness/flexibility of the ring about any desired axis. Of course, other methods of modifying the ring material as disclose herein are similarly diverse.

The terms and expressions which have been used in the description above are used only as terms of description and not of limitation. There is no intention of excluding equivalents of the features shown or described. It is recognized that one having ordinary skill in this art would perceive equivalence to the inventions claimed below, which equivalence would be within the spirit of the invention as expressed above.

What is claimed is:

1. A device for correcting refractive defects of the vision comprising: a biocompatible implant for implantation within the cornea of an eye, said biocompatible implant having at least one region with a modified bulk or apparent or bulk and apparent modulus of elasticity.

2. The device of claim 1 wherein said biocompatible implant has an overall cross sectional area which is substantially constant throughout said biocompatible implant.

3. The device of claim 1 wherein said at least one region of modified bulk or apparent or bulk and apparent modulus of elasticity comprises a material having a greater bulk or apparent or bulk and apparent modulus of elasticity than the remainder of said biocompatible implant.

4. The device of claim 1 wherein said at least one region of modified bulk or apparent or bulk and apparent modulus of elasticity comprises a composite material having a greater apparent modulus of elasticity than the bulk or apparent or bulk and apparent modulus of elasticity of a remainder of said biocompatible implant.

5. The device of claim 1 wherein said biocompatible implant comprises a ring having two regions of modified bulk or apparent or bulk and apparent modulus of elasticity having greater flexibility than a remainder of said biocompatible ring.

6. The device of claim 5 wherein said two regions of modified bulk or apparent or bulk and apparent modulus of elasticity comprise a material having a lower bulk or apparent or bulk and apparent modulus of elasticity than the bulk or apparent modulus or bulk and apparent modulus of elasticity of said remainder of said biocompatible ring.

7. The device of claim 5 wherein said two regions of modified bulk or apparent or bulk and apparent modulus of elasticity comprise a composite material having a lower apparent modulus of elasticity than the bulk or apparent or bulk and apparent modulus of elasticity of said remainder of said biocompatible ring.

8. The device of claim 1 wherein said at least one region of modified bulk or apparent or bulk and apparent modulus of elasticity has at least one hole therethrough to reduce the modulus thereof.

9. The device of claim 8 wherein said at least one hole is filled with a material having a lower bulk or apparent modulus of elasticity than said at least one region in which said at least one hole is formed.

10. The device of claim 1 wherein said at least one region of modified bulk or apparent or bulk and apparent modulus of elasticity has at least one feature for modifying the stiffniess of said at least one region, said at least one feature selected from the group consisting of grooves, dimples, voids, gas bubbles, tortuous cavities, low modulus inclusions and radiation exposed regions.

11. The device of claim 1 wherein said biocompatible implant comprises a split ring having two regions of modified bulk or apparent or bulk and apparent modulus of elasticity having greater flexibility than a remainder of said split ring.

12. The device of claim 1 wherein said biocompatible implant comprises a partial ring having a gap separating ends of said partial ring.

13. The device of claim 1 wherein said biocompatible implant comprises at least one segment of a ring.

14. The device of claim 13, wherein said at least one segment of a ring comprises a pair of segments which, when implanted approximate the shape of a ring.

15. The device of claim 14, wherein each of said segments comprises at least one region of modified bulk or apparent or bulk and apparent modulus of elasticity.

16. An intracorneal implant comprising:
at least two zones circumferentially defined about said implant; wherein one of said at least two zones has elasticity which is substantially different from an elasticity of at least one of the other of said at least two zones.

17. The intracomeal implant of claim 16, wherein said at least two zones comprises four zones, wherein two of said zones diametrically oppose one another and have substantially the same elasticity.

18. The intracomeal implant of claim 17, wherein the other two of said four zones diametrically oppose one another and have substantially the same elasticity.

19. The intracorneal implant of claim 17, wherein said two of said zones are relatively more flexible than the remainder of said implant, said implant being foldable along said two of said zones to reduce the area of said implant during implantation.

20. The intracorneal implant of claim 16, wherein said at least two zones are joined by a butt joint.

21. The intracorneal implant of claim 16, wherein said at least two zones are joined by a lap joint.

22. The intracomeal implant of claim 16, wherein said at least two zones are joined by a tapered joint.

23. The intracorneal implant of claim 16, wherein at least one of said at least two zones contains holes for varying the flexibility thereof.

24. The intracomeal implant of claim 16, wherein said at least two zones have substantially equal thicknesses.

25. The intracorneal implant of claim 16, wherein said at least two zones have substantially equal bulks.

26. A method for forming an intracorneal implant having at least one zone of modified elasticity, said method comprising:
coextruding a ribbon of material having at least two bands of material with varying elastic moduli; and
punching out a body from the ribbon, such that the body includes portions of each of the at least two bands of material.

27. The method of claim 26, further comprising:
thermoforming the body to set the body to have a predetermined cone angle.

28. The method of claim 26, wherein the ribbon comprises three bands including a middle band having a relatively rigid modulus of elasticity, surrounded by two bands having relatively lower elastic moduli than the middle band.

29. The method of claim 26, wherein the ribbon comprises three bands including a middle band having a relatively flexible modulus of elasticity, surrounded by two bands having relatively higher elastic moduli than the middle band.

30. A device for correcting refractive defects of the vision comprising: a biocompatible implant for implantation within the cornea of an eye, said biocompatible implant having a substantially constant cross-sectional area and varying elasticity.

31. A method for forming an intracorneal implant having at least one zone of modified elasticity, said method comprising:
co-casting at least two materials having different moduli of elasticity into a shape of said intracorneal implant.

32. A method for forming an intracorneal implant having at least one zone of modified elasticity, said method comprising:
modifying at least one zone of the intracorneal implant to change the modulus of elasticity of the at least one zone to be diverse from the modulus of elasticity of an adjacent zone of the intracorneal implant.

33. The method of claim 32, wherein said modifying comprises forming at least one hole in said at least one zone.

34. The method of claim 32, wherein said modifying comprises forming at least one void in said at least one zone.

35. The method of claim 32, wherein said modifying comprises forming at least one groove in said at least one zone.

36. The method of claim 32, wherein said modifying comprises forming at least one dimple in said at least one zone.

37. The method of claim 32, wherein said modifying comprises forming at least one gas bubble in said at least one zone.

38. The method of claim 32, wherein said modifying comprises forming at least one tortuous cavity in said at least one zone.

39. The method of claim 32, wherein said modifying comprises forming at least one inclusion, having a modulus of elasticity which is diverse from the at least one zone, in said at least one zone.

40. The method of claim 32, wherein said modifying comprises exposing said at least one zone to radiation.

41. The method of claim 32, wherein said modifying comprises exposing said at least one zone to a solvent.

42. The method of claim 32, wherein said modifying comprises exposing said at least one zone to a plasticizer.

43. A device for correcting refractive defects of the vision comprising: a biocompatible implant for implantation within the cornea of an eye, said biocompatible implant having at least one region having a modified modulus of elasticity, each said at least one region subtending an angle of at least about 30°.
